(19) United States
(12) Patent Application Publication
(10) Pub. No.: US 2025/0025334 A1
(43) Pub. Date: Jan. 23, 2025

(54) DEVICES FOR IMPLANTATION OF CELLULAR TRANSPLANTS AND METHODS OF USING THE SAME

(71) Applicant: The Regents of the University of Colorado, a body corporate, Denver, CO (US)

(72) Inventors: Marc Mathias, Denver, CO (US); Kang Li, Baltimore, MD (US); Miguel Flores Bellver, Centennial, CO (US); Silvia Aparicio Domingo, Centennial, CO (US); Conan Chen, Denver, CO (US); Bruce Beckstein, Sarasota, FL (US); Segundo Rodriguez, Sarasota, FL (US)

(21) Appl. No.: 18/784,462

(22) Filed: Jul. 25, 2024

Related U.S. Application Data
(63) Continuation of application No. 18/627,279, filed on Apr. 4, 2024, now abandoned.
Application No. 18/627,279 is a continuation of application No. PCT/US22/45572, filed on Oct. 3, 2022.
(60) Provisional application No. 63/280,991, filed on Nov. 18, 2021.
(60) Provisional application No. 63/252,129, filed on Oct. 4, 2021.

(51) Int. Cl. A61F 9/00; A61B 17/00; A61B 17/30

(52) U.S. Cl. CPC A61F 9/0017; A61B 17/30; A61B 2017/00469; A61B 2017/305

(57) ABSTRACT

Embodiments disclosed herein concern a device for implantation of a cellular transplant into a target region of a subject. The device includes: a handpiece; a shaft anchored with the handpiece and extending distally therefrom; and a tubular sheath. The shaft includes a proximal end, a distal end opposite the proximal end, a curved portion, and forceps at the distal end thereof. The forceps include a pair of distal tips configured to releasably grasp the cellular transplant. The tubular sheath is movably coupled to and extending distally from the handpiece. The tubular sheath includes a distal opening and a lumen configured to slidably receive the shaft and the forceps therein. In a non-deployed state, the distal tips of the forceps are positioned within the lumen of the tubular sheath, and, in a deployed state, the distal tips of the forceps are positioned partially outside the distal opening of the tubular sheath.

### CROSS-REFERENCE TO RELATED APPLICATIONS

This application is a continuation application of U.S. application Ser. No. 18/627,279 filed Apr. 4, 2024, which is a continuation application of International Application No. PCT/US2022/045572, filed Oct. 3, 2022, which claims priority under 35 U.S.C. § 119 (e) to U.S. Provisional Application No. 63/252,129, filed Oct. 4, 2021, and U.S. Provisional Application No. 63/280,991, filed Nov. 18, 2021. These applications are incorporated herein by reference in their entireties for all purposes.

### FIELD

Embodiments of the instant disclosure relate to devices and methods of using the same for harvesting and cellular implantation in the eye of a subject in need thereof.

### BACKGROUND

Retinal pigment epithelium (RPE) plays an important role in supporting normal photoreceptor function. RPE damage can lead to secondary dysfunction and degeneration of photoreceptor cells, which in turn can cause severe, irreversible vision impairment in patients affected by conditions such as age-related macular degeneration (AMD) and Stargardt's disease. Recently, clinical trials involving transplantation of embryonic stem cell-derived RPE in patients with AMD showed promising safety and efficacy outcomes. Moreover, RPE derived from human-induced pluripotent stem cells (hiPSC), which could be utilized for autologous therapies, is also being evaluated in clinical trials. Accordingly, subretinal delivery devices for preservation of harvested or cultured cells such as stem cells and preservation and delivery of RPE single cells and/or monolayers of RPE cells or tissue are needed in this advancing field.

### SUMMARY

Embodiments disclosed herein generally relate to devices for cellular implantation of the eye. In certain embodiments, cellular implantations concern supplementing native cells, inducing repair of native cells, or replacing some, or all the cells or tissues that make up retinal pigment epithelium (RPE). In some embodiments, a device for implantation of single cells, a plurality of cells, monolayers of cells and/or cellular tissue into a target region is described. In accordance with these embodiments, the devices include, but are not limited to, a handpiece; a curved tubular sheath extended in a distal direction from the handpiece; a shaft anchored with the handpiece, the shaft including a curved first portion that is parallel to and located inside the curved tubular sheath; and a second portion located outside the distal end of the curved tubular sheath and further including micro-jaws at its distal end; and a movement control system configured to cause longitudinal movement of the curved tubular sheath relative to the handpiece and shaft. In some embodiments, the longitudinal movement of the curved tubular sheath causes the sheath to slide over the micro-jaws to facilitate the micro-jaws to close and grasp the cellular transplant from a transplant carrier.

Some embodiments disclosed herein concern methods of implanting single cells and/or monolayers of cellular tissue into an eye of a subject recipient. In accordance with these methods, the implantation device disclosed herein can be utilized to perform the methods. In certain embodiments, the method include, but are not limited to, providing the cellular implant or tissue device and using the micro-jaws of the device to grasp the single cells, multiplicity of cells, monolayers and/or cellular tissue from the subject to be treated or a transplant donor; inserting a segment or part of the curved tubular sheath of the device into the eye of the subject recipient; and releasing the cellular transplant inside the eye of the subject recipient. In other embodiments, prior to the inserting the segment or part of the curved tubular sheath, an incision is made in the eye of the subject. In certain embodiments, the incision can be about a width of about 1 mm to about 2 mm.

Other embodiments disclosed herein concern surgical kits containing at least one cellular implant device disclosed herein and instructions for a user to operate the device. Some embodiments concern kits having at least one container for storage, transport and use of devices disclosed herein.

Certain aspects of the present disclosure can include devices for implantation of a cellular or cell transplant or cell implant into a target region. The cell transplant can include one or more cells and, in some embodiments, the one or more cells can be on substrate. In accordance with these aspects, the devices can include: a handpiece; a shaft anchored with the handpiece and extending distally therefrom; and a tubular sheath. The shaft can include a proximal end, a distal end opposite the proximal end, a curved portion, and forceps at the distal end thereof. The forceps can include a pair of distal tips configured to releasably grasp the cellular transplant. The tubular sheath can be movably coupled to and extending distally from the handpiece. The tubular sheath can include a distal opening and a lumen configured to slidably receive the shaft and the forceps therein. In a non-deployed state, the distal tips of the forceps are positioned within the lumen of the tubular sheath, and, in a deployed state, the distal tips of the forceps are positioned at least partially outside the distal opening of the tubular sheath.

In certain embodiments, retraction of the tubular sheath relative to the handpiece transitions the device from the non-deployed state to the deployed state.

In certain embodiments, in the non-deployed state, the forceps are configured to grasp the cellular transplant, and, in the deployed state, the forceps are configured to release the cellular transplant.

In certain embodiments, in the non-deployed state, the forceps are compressed by the tubular sheath to permit grasping the cell transplant or cell implant.

In certain embodiments, the cellular implant can be configured to transition from a folded orientation to an essentially flat orientation when the device transitions from the non-deployed state to the deployed state.

In certain embodiments, the distal opening of the tubular sheath is beveled. In certain embodiments, the tubular sheath is beveled by or at an angle of about 10 degrees to about 50 degrees. In certain embodiments, the tubular sheath is beveled by about 15 degrees. In certain embodiments, the tubular sheath is beveled by an angle of about 25 degrees. In certain embodiments, the tubular sheath is beveled by an angle of about 35 degrees. In certain embodiments, the tubular sheath is beveled by an angle of about 45 degrees.

In certain embodiments, the device further includes a seal positioned between the shaft and an inner wall of the tubular sheath, the seal preventing backflow of fluid into the lumen of the tubular sheath.

In certain embodiments, the forceps are non-locking forceps including a hinge opposite the distal tips.

In certain embodiments, the shaft includes a curved portion, and the curved portion of the shaft can include a compound curve.

In certain embodiments, the shaft includes a curved portion, and the curved portion of the shaft is configured to angle the forceps at an upward angle relative to the handpiece.

In certain embodiments, the shaft includes a curved portion, and the curved portion of the shaft matches a profile of a posterior pole of an eye.

In certain embodiments, the shaft includes a curved portion, and the curved portion of the shaft orients the forceps at about a forty-five degree angle relative to a longitudinal axis of the handpiece.

In certain embodiments, the device further includes an actuator coupled with the tubular sheath and moveably coupled to the handpiece, whereby actuation of the actuator is configured to cause retraction of the tubular sheath relative to the handpiece permitting transition of the device from the non-deployed state to the deployed state.

In certain embodiments, the actuator can include a rotatable member in which rotation of the rotatable member causes retraction of the tubular sheath relative to the handpiece.

In certain embodiments, the shaft is rigid, and the tubular sheath is flexible to permit the tubular sheath to conform to a shape of the shaft as it slides over the shaft. In certain embodiments, the tubular sheath is translucent. In certain embodiments, the tubular sheath can include a Teflon material or other suitable material such as another material that is flexible, is biocompatible, and capable of sliding over the shaft.

In certain embodiments, the cell transplant is made up of one or a plurality of cells. In certain embodiments, the cell transplant is made up of one or a plurality of cells on a substrate. In another embodiment, the cell transplant can include a retinal pigment epithelium (RPE) cellular transplant, a cellular transplant including retinal cells, or a retina-RPE complex transplant. In other embodiments, a cellular transplant includes a cell adhesion layer or substance that permits cells to adhere for example as a monolayer and further includes one or more cells.

Aspects of the present disclosure can include a surgical kit including the device described herein and instructions for use. In certain embodiments, the surgical kit can also include a trephine configured to cut the cellular implant. In some embodiments, the trephine is configured to cut the cellular implant in a shape that can include a circular portion and a tab portion extending outward from a perimeter of the circular portion. In certain embodiments, the surgical kit can also include a carrier device including a carrier housing and a carrier plate configured to be inserted into the carrier housing, the carrier device configured to house the cellular implant.

In other embodiments, kits contemplated herein can further include a container for storing or transporting the surgical kit described herein.

Aspects of the present disclosure can include methods for implanting a cellular transplant into an eye of a subject. In accordance with these embodiments, the methods can include: providing a device according to the present disclosure; using the forceps of the device to grasp the cellular transplant; inserting a portion of the tubular sheath of the device into the eye of the subject; and releasing the cellular transplant inside the eye of the subject.

In certain embodiments, the methods can further include, prior to the inserting the portion of the tubular sheath, making an incision in the eye of the subject that is smaller than a diameter of the cellular transplant.

In certain embodiments, releasing the cellular transplant inside the eye of the subject can include retracting the tubular sheath relative to the handpiece so as to unfold the cellular transplant and releasing the forceps from grasping the cellular transplant inside the eye.

In certain embodiments, the cellular transplant is released within a subretinal region of the eye of the subject. In certain embodiments, the cellular transplant can include a retinal pigment epithelium (RPE) cellular transplant, a retina-RPE complex transplant, a neural retina cellular transplant, or a neural retina-RPE cellular transplant.

In certain embodiments, the methods can further include, prior to or during the release of the cellular transplant, causing no or minimal fluid reflux into the tubular sheath of the device.

In certain embodiments, the subject has a retinal condition. In accordance with these embodiments, the retinal condition can include, but is not limited to, one or more of a retinal degenerative condition or a retinal injury, the retinal condition causing loss or damage to the retinal pigment epithelium (RPE) or outer neurosensory retina, or a combination thereof.

In certain embodiments, the retinal condition can include one or more of advanced dry age-related macular degeneration, (AMD), retinitis pigmentosa, Stargardt disease, Best disease, Sorsby's fundus dystrophy, Doyne honeycomb retinal dystrophy, retinal trauma, or a combination thereof.

In certain embodiments, the subject is a human. In other embodiments, the subject is not human but can be an animal or other subject experiencing a health condition of the eye in need of cellular transplantation. It is contemplated that the devices disclosed herein are scalable in order to be used to deliver a cellular implant to any subject contemplated.

### BRIEF DESCRIPTION OF THE FIGURES

FIG. 1A illustrates a retinal pigment epithelium cell spheroid from retinal organoids according to certain embodiments disclosed herein.

FIG. 1B illustrates the characteristic pigmentation, hexagonal morphology, and intact F-actin cytoskeleton of the RPE cells after a period of time in culture according to certain embodiments disclosed herein.

FIG. 1C illustrates intact F-actin cytoskeleton (Phalloidin) and proper localization of premelanosome protein (PMEL17) in iPSCs (human, hiPSC-derived RPE) monolayers after a period of time in culture according to certain embodiments disclosed herein.

FIG. 1D illustrates a healthy and functionally mature state of iPSCs (human, hiPSC-derived RPE) tissue. Certain biomarkers demonstrate healthy control expression levels in the iPSCs in culture according to certain embodiments disclosed herein.

FIG. 2A is a block diagram of exemplary steps in preparing, storing, transporting, and implanting a subretinal implant into a patient eye according to certain embodiments disclosed herein.

FIG. 2B is a side view of a trephine having a distal cutting tip that outlines the shape of the cell transplant according to certain embodiments disclosed herein.

FIG. 3A is a top view of a cell transplant, the shape of which is defined by the cut made by the distal cutting tip of the trephine of FIG. 2B according to certain embodiments disclosed herein.

FIG. 3B are isometric top views of a cell transplant carrier and its component pieces, a carrier housing or chest and a carrier plate or drawer according to certain embodiments disclosed herein.

FIG. 3C is a close-up top view of a chamber of the carrier plate with a cell transplant, shown in dotted line, positioned within the chamber according to certain embodiments disclosed herein.

FIG. 4A-4H are, respectively, a front isometric view, a front isometric exploded view, a first side view, a second side view (opposite the first side view), a front/distal view, a back/proximal view, a top view, and a bottom view of a surgical implantation device according to certain embodiments disclosed herein.

FIGS. 4I and 4J are, respectively, an enlarged isometric view of the surgical implantation device and an enlarged cross-sectional side view of the surgical implantation device according to certain embodiments disclosed herein.

FIG. 4K is an enlarged cross-sectional side view of the forceps and tubular sheath of the surgical implantation device according to certain embodiments disclosed herein.

FIG. 4L depicts a pair of side views of a distal end of a surgical implantation device. In the top image, the device has a seal and, in the bottom image, the device is without a seal. Both devices are in the deployed state with the distal tips of the forceps exposed out from the distal opening of the tubular sheath for deploying the cellular transplant for implantation at an implantation site according to certain embodiments disclosed herein.

FIG. 4M is a side view of the surgical implantation device illustrating various exemplary dimensions of the device. The device is positioned relative to a diagram of an eye to demonstrate the relative size of the device according to certain embodiments disclosed herein.

FIGS. 4N-4Q illustrate subsequent steps of grasping of a cell transplant by the forceps of the surgical implantation device and the housing of the cell transplant in the lumen of the tubular sheath according to certain embodiments disclosed herein.

FIG. 4R is a flowchart illustrating an exemplary method of performing a surgery using the surgical implantation device described herein.

FIG. 4S illustrates a side view of the surgical implantation device positioned relative to an eye according to certain embodiments disclosed herein.

### DETAILED DESCRIPTION

Embodiments of the present disclosure provides for devices for harvesting, transporting, and inserting cell transplants, cellular substrates, cellular implants or cell-containing tissues into the eye of a subject in need thereof.

It is noted that as used herein and in the appended claims, the singular forms “a”, “an”, and “the” include plural reference unless the context clearly dictates otherwise. Thus, for example, reference to “an appliance” includes a plurality of such appliances and equivalents thereof known to those skilled in the art, and so forth. As well, the terms “a” (or “an”), “one or more” and “at least one” can be used interchangeably herein. It is also to be noted that the terms “comprising”, “including”, and “having” can be used interchangeably.

Number ranges are to be understood as inclusive, i.e. including the indicated lower and upper limits. Furthermore, the term “about”, as used herein, and unless clearly indicated otherwise, generally refers to and encompasses plus or minus 10% of the indicated numerical value(s). For example, “about 10%” can indicate a range of 9% to 11%, and “about 1” can include the range 0.9-1.1.

The term “biocompatible,” as used herein, refers to a material that does not elicit an immunological rejection or detrimental effect, referred herein as an adverse immune response when it is disposed within an in vivo biological environment. For example, in certain embodiments a biological marker indicative of an immune response to a material changes less than 10%, or less than 20%, or less than 25%, or less than 40%, or less than 50% from a baseline value when a human or animal is exposed to or in contact with the biocompatible material. Alternatively, immune responses can be determined histologically, where a localized immune response can be assessed by visually assessing markers (e.g., through binding assays etc.), including visualizing immune cells or markers that are involved in an immune response pathway, in and adjacent to the material such as a localized immune response. In one aspect, a biocompatible material or device does not observably adversely change immune response as determined histologically. In some embodiments, the disclosure provides biocompatible devices configured for long-term use, for example, on the order of weeks to months, without invoking an adverse immune response. In certain embodiments, biological effects can be initially evaluated by measurement of cytotoxicity, sensitization, irritation and intracutaneous reactivity, acute systemic toxicity, pyrogenicity, subacute/subchronic toxicity and/or implantation. Biological tests for supplemental evaluation disclosed herein can include testing for chronic toxicity.

“Bioinert” refers to a material that does not elicit an immune response from a human or animal when it is disposed within an in vivo biological environment. For example, a biological marker indicative of an adverse immune response remains essentially constant (e.g., plus or minus 5% of a baseline value) when a human or animal is exposed to or in contact with the bioinert material. In some embodiments, the disclosure provides bioinert devices.

All publications, patents, and patent applications mentioned in this specification are herein incorporated by reference to the same extent as if each individual publication, patent, or patent application was specifically and individually indicated to be incorporated by reference.

Embodiments disclosed herein generally relate to devices for cellular implantation of the eye. For the surgical implantation devices, in addition to the biocompatible and bioinert definitions provided herein, the material for the devices does not cause any direct toxicity to the cell transplant including the cells and/or tissues and/or cellular substrate being stored and/or transported (e.g., does not cause the cells to die, or trigger cellular abnormalities, etc.). In certain embodiments, surgical implantation devices described herein can be manufactured from materials that have very little or that do not have any adverse/toxic effect on the cells or cellular substrate of the cell transplant being implanted in a subject with the device.

In certain embodiments, cellular implantations concern supplementing current or the naturally-occurring cells of a subject, inducing repair of current or naturally-occurring cells or replacing some or all of the cells or tissues of a subject that make up RPE. In some embodiments, a device for implantation of single cells and/or monolayers of cellular tissue or a cellular substrate of a cellular transplant into a target region is described. In accordance with these embodiments, the devices include, but are not limited to, a handpiece; a curved tubular sheath extended in a distal direction from the handpiece; a shaft anchored with the handpiece, the shaft including a curved first portion that is parallel to and located inside the curved tubular sheath; and a second portion located outside the distal end of the curved tubular sheath and further including micro-jaws at its distal end; and a movement control system configured to cause longitudinal movement of the curved tubular sheath relative to the handpiece and shaft. In some embodiments, the longitudinal movement of the curved tubular sheath causes the sheath to slide over the micro-jaws to facilitate the micro-jaws to close and grasp the cellular transplant from a transplant carrier.

Other embodiments of devices disclosed herein include a feature where the longitudinal movement of the curved tubular sheath causes the cellular transplant to retract into the sheath lumen. Other embodiments concern a feature where the cellular transplant that is attached to the closed micro-jaws is caused to fold concavely into the lumen of the sheath.

Some embodiments concern devices where the movement control system includes a wheel configured to move forward or backward causing the curved tubular sheath to advance or retract, respectively. Other movement control systems can be configured to release the single cells and/or monolayers of cellular tissue, or cellular transplant into a target region of a subject with a controlled delivery speed. In certain embodiments, the target region includes the eye of the subject.

In certain embodiments, the curved tubular sheath is translucent. In some embodiments, the curved tubular sheath is made of a Teflon or Teflon blend material or material resistant to rust or other corrosion that can be caused by exposure to fluids of the eye or other moisture contact.

In other embodiments, the distal end of the curved tubular sheath is beveled. In accordance with these embodiments, the distal end of the curved tubular sheath can include about a 15° to about a 30° bevel.

In certain embodiments, a target of an eye of a subject is the retina. In some embodiments, the curved portion of the tubular sheath is configured to fit the curve of the posterior pole of the retina. In other embodiments, a cellular transplant can include a retinal pigment epithelium (RPE) cellular transplant or a retina-RPE complex transplant.

In other embodiments, the handpiece is ergonomic in design and has a length of about 70 mm to about 100 mm. In other embodiments, a curved tubular sheath of a device disclosed herein can have a diameter of about 0.5 mm to about 1.5 mm. In other embodiments, the handpiece can be adjusted or created in size to suitably fit the user such as the health professional.

In some embodiments, devices disclosed herein can be part of a kit.

In other embodiments, devices disclosed herein can be used to treat an eye condition of a subject. In accordance with these embodiments, devices disclosed herein can be used to treat any eye condition by making an incision in an eye of a subject having a width of about 1 mm to about 2 mm and inserting a cell, a cellular implant, cellular transplant (with or without a cellular substrate) or cellular tissue into the subject's eye. In some embodiments, the device carries or contains harvested cells and then releases single cells, a multitude of cells and/or one or more monolayer of cells or cellular tissue inside the eye by retracting the curved tubular sheath, unfolding the cellular transplant into an initial shape, and opening the micro-jaws to release the single cells, a multitude of cells and/or one or more monolayer of cells or cellular tissue inside the eye. In some embodiments, the single cells, a multitude of cells and/or one or more monolayer of cells or cellular tissue are released within a subretinal region of the eye. In other embodiments, a health profession can visualize the single cells, a multitude of cells and/or one or more monolayer of cells or cellular tissue during the insertion and release steps. In some embodiments, little to no fluid reflux in the eye occurs.

In some embodiments, the subject has an eye condition. In certain embodiments, the eye condition includes, but is not limited to, a retinal degenerative condition. In other embodiments, the retinal degenerative condition includes, but is not limited to, one or more of advanced dry age-related macular degeneration, (AMD), retinitis pigmentosa, Stargardt disease, Best disease, Sorsby's fundus dystrophy, Doyne honeycomb retinal dystrophy or a combination thereof.

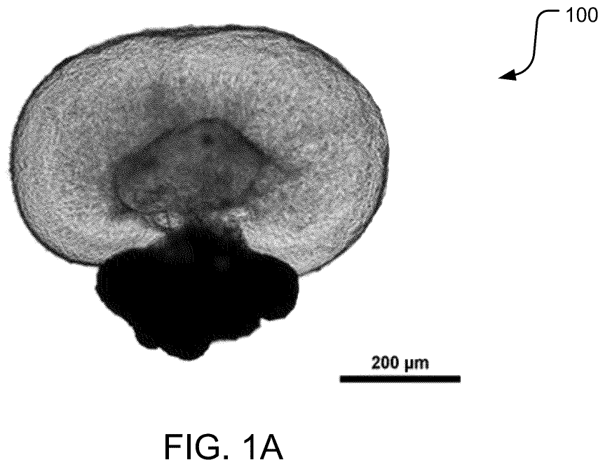

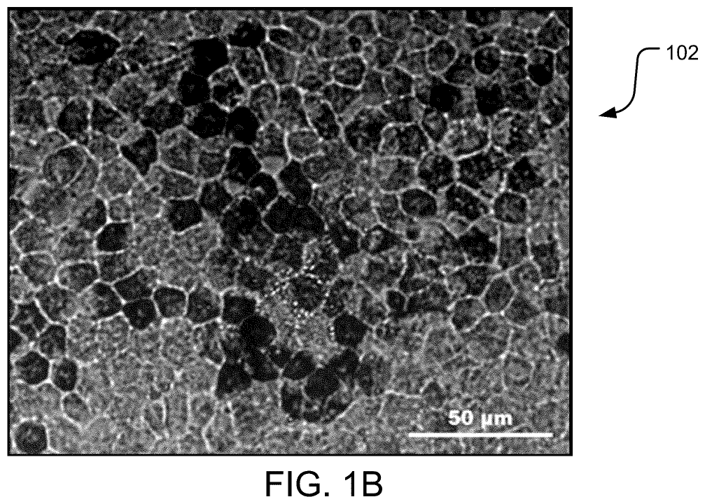

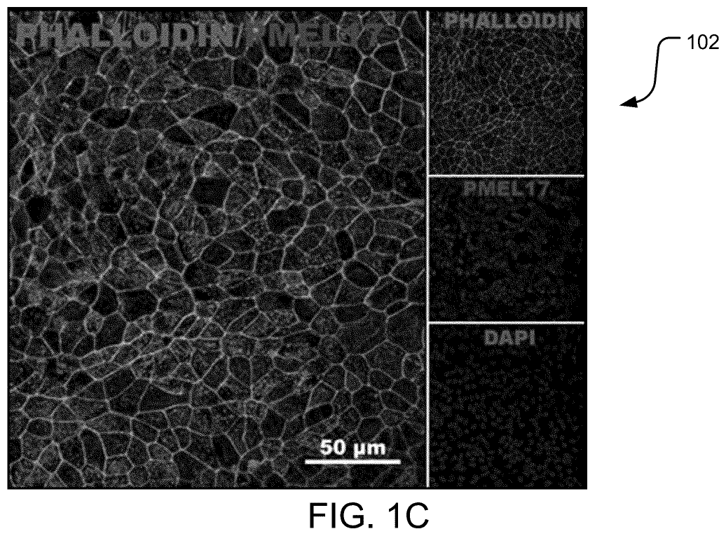

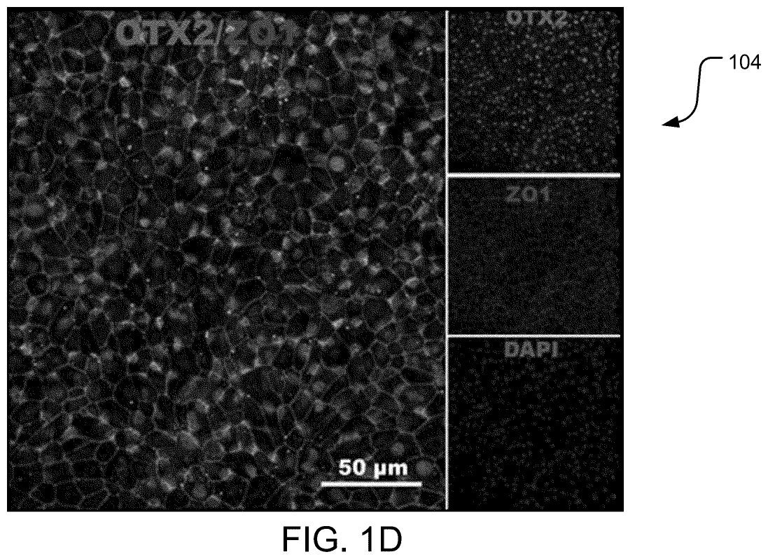

With reference to FIG. 1A, which illustrates retinal pigment epithelium (RPE) spheroids from retinal organoids 100, an embodiment of the present disclosure can include generating human-induced pluripotent stem cells (hiPSC) derived RPE monolayers to use as cellular substrates for an RPE implant. The RPE spheroids from the retinal organoids of FIG. 1A can be harvested and cultured on, for example, 10 μm thick transparent polyester membranes. After a period of time (e.g., 40 days of differentiation), hiPSC-RPE cells 102 showed the characteristic pigmentation, typical hexagonal morphology, and intact F-actin cytoskeleton, which can be seen in FIGS. 1B-1C.

In certain embodiments, the hiPSC-RPE cells can be characterized by the expression of key marker proteins involved in normal RPE cell differentiation and function, including, but not limited to, premelanosome protein (PMEL17), orthodenticle homeobox 2 (OTX2), and zonula occludens-1 (ZO-1). While PMEL17 is known to be enriched in premelanosomes, OTX2 aids in differentiation of RPE cells and transactivation of the genes involved in melanosome formation, and ZO-1 is a membrane-associated tight junction adaptor protein that links junctional membrane proteins to the cytoskeleton and plays an important role in RPE homeostasis in vivo. In accordance with these embodiments, a healthy control level of expression of PMEL17, OTX2, and ZO-1 can confirm that hiPSC-RPE tissue 104 is in a healthy and functionally mature state, as observed for example, in FIG. 1D.

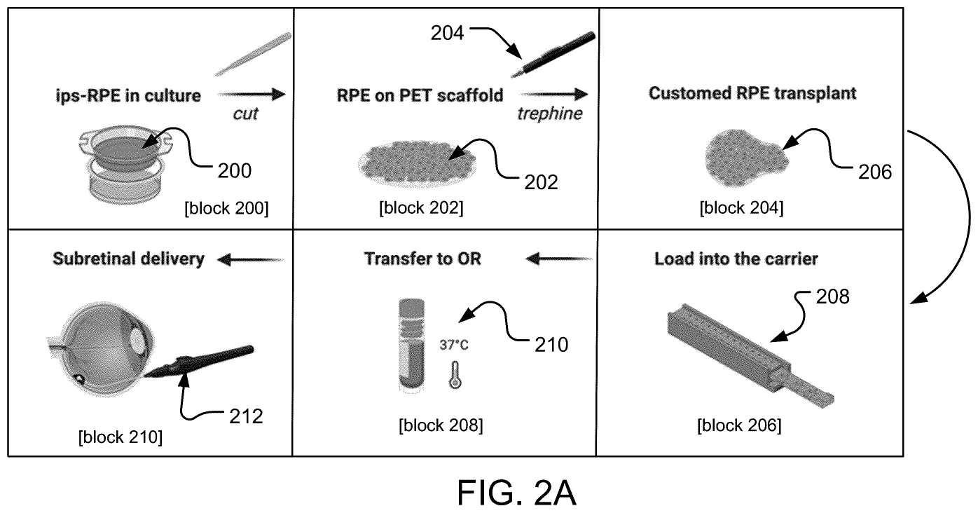

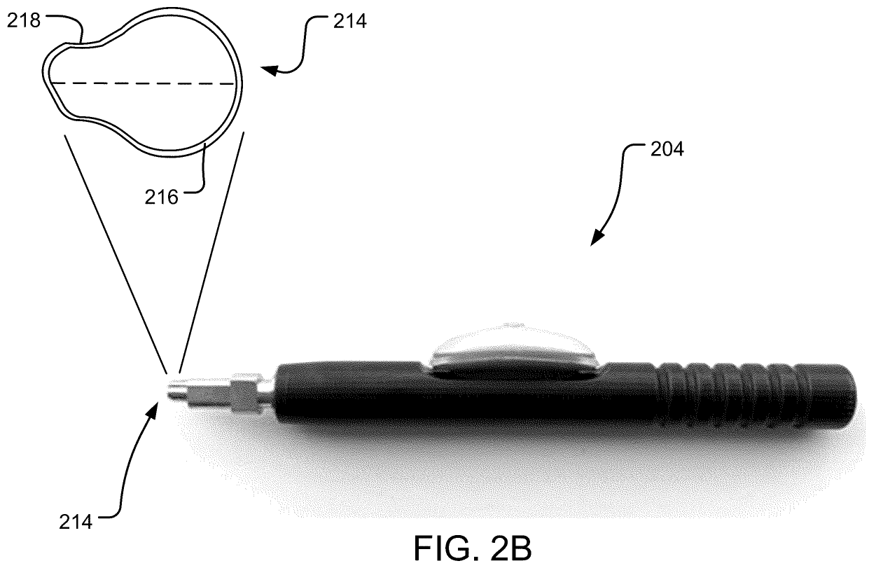

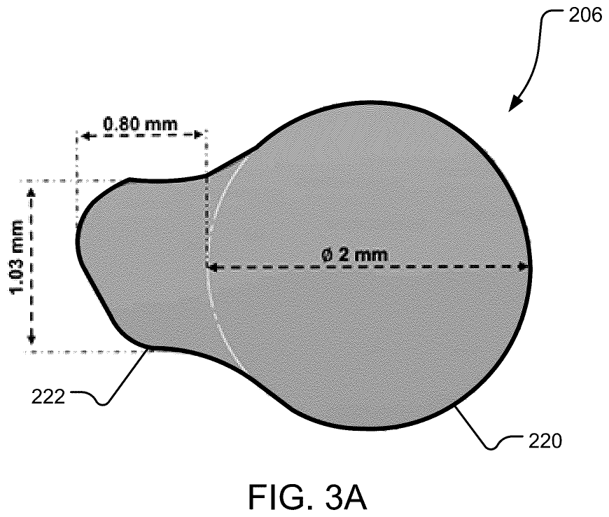

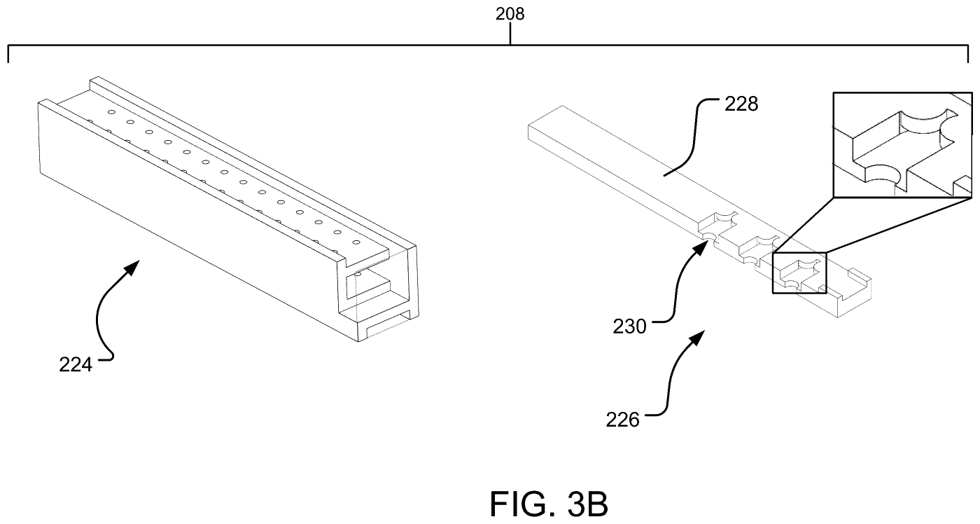

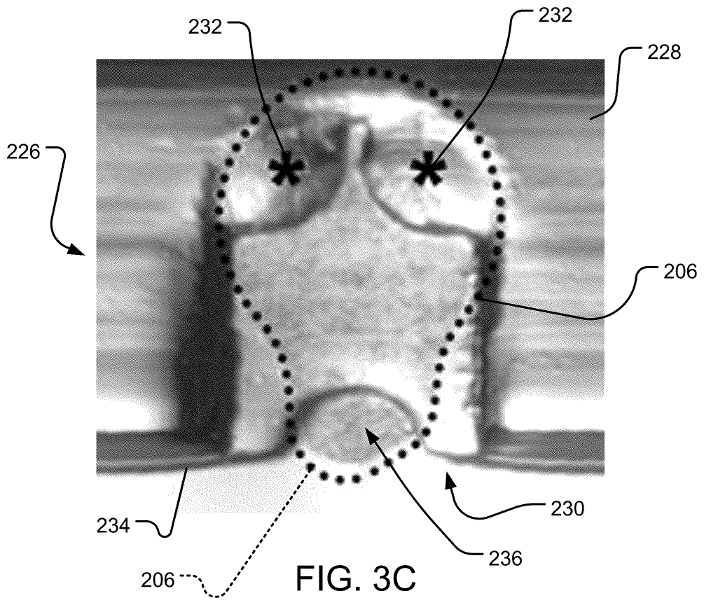

One embodiment of cellular transplant carrier devices and methods is described with reference to FIGS. 2A-3C. The cellular transplant carrier device can be referred to as a cellular or cell transplant carrier device. To begin, reference is made to FIG. 2A, which illustrates an overview of various stages in the production, transportation, and implantation of transplants, such as hiPSC-RPE transplants. As observed in [block 200] of FIG. 2A, cell cultures 200 are removed from a dish or tray and placed on a PET scaffold. From the removed cell cultures on a PET scaffold 202, illustrated in [block 202], a trephine or punch 204 (illustrated in further detail in FIG. 2B) is used to obtain custom sized cell transplant (which, in certain embodiments, can include a cell substrate) 206 (illustrated in further detail in FIG. 3A). As illustrated in [block 206], the cell transplant 206 are loaded into a cell transplant carrier 208 (shown in further detail in FIGS. 3B-3C), which is designed to safely hold the cell transplants 206. The cell transplant carrier 208 can then be loaded into a carrier or chamber 210 (e.g., tube) for transportation to an operating room, as seen in [block 208]. Then, as seen in [block 210] of FIG. 2A, the cell transplant carrier 208 can be removed from the chamber 210, and a delivery tool 212 can be utilized to obtain a cell transplant 206 from the carrier 208 and use it in a surgical procedure.

Turning to FIG. 2B, the trephine 204 is illustrated in a side view. The trephine 204 includes a handle and a distal cutting tip 214 that outlines the shape of the cell transplant 206. As illustrated in the figure, the distal cutting tip 214 includes a circular portion 216 on the right in the enlarged image of FIG. 2B, and an asymmetrical tab portion 218 extending to the left of the circular portion. The tab 218 is asymmetrical in that it is not symmetric along the longitudinal axis (shown by illustration as a dotted line). Instead, it includes a larger or prominent portion at the top and a smaller or inferior portion at the bottom. The asymmetric nature provides visual confirmation of the orientation of the cell transplant 206. For instance, in embodiments with cells on a substrate, there will be a cell-side and a substrate-side. With the asymmetric nature of the tab 218 it can be visually verified if the correct side of the cell transplant 206 is being grasped and implanted.

FIG. 3A depicts a top view of the cell transplant 206. The shape of the cellular transplant 206 is defined by the cut made by the distal cutting tip 214 of the trephine 204 in FIG. 2B. Like the distal tip 214 of the trephine 204, the cellular transplant 206 has a circular portion 220 and an asymmetrical tab portion 222 that includes a larger portion (upper end of image) and a smaller portion (lower end of image). In this way, the orientation of the cellular transplant 206 is readily apparent (cell side up or upside down) by observing the asymmetrical tab portion 222 of the transplant 206. For example, the cellular transplant 206 can be cell side up when the larger portion of the asymmetrical tab portion 222 is positioned in a clockwise direction from the smaller portion when the transplant 206 is viewed from above. This is merely an example, and the cellular transplant 206 can be differently (e.g., oppositely) configured without limitation.

Exemplary dimensions for the cellular transplant 206 are illustrated in the figure, with a diameter of the circular portion being about 2 mm. A width of the tab portion 222 can be about 1.03 mm. A length of the tab portion 222 can be about 0.80 mm. These dimensions are exemplary for one embodiment of a hiPSC-RPE transplant. The dimensions can be modified given the type of implant and the biological environment for the transplant.

The tab portion 222 is configured to be grasped by a delivery instrument. The circular portion 220 is to be minimally disturbed during loading into the cell transplant carrier 208, which is illustrated in FIG. 3B. In one example, the cell transplant carrier 208 is illustrated in its component pieces, which include the carrier housing or chest 224 and the carrier plate or drawer 226. The carrier plate 226 includes a body 228 and chambers 230 defined in the body 228. Each of the chambers is sized and shaped to receive the cell substrate or transplant 206 shown in FIG. 3A. When the cellular transplant 206 is received in the chamber 230, the cellular transplant 206 is secured in place within the chamber 230 while being partially exposed in order to permit removal from the chamber 230, as will be described subsequently. The carrier housing 224 includes a carrier plate opening 232 leading to an inner compartment configured to removably receive the carrier plate 226. In this example, the carrier plate 226 illustrated in FIG. 3B illustrates the plate 226 having three chambers 230. However, the plate 226 can include more or fewer chambers 230. For example, the plate 226 can include a single chamber 230. In other embodiments, the plate 226 can include two chambers 230. In other embodiments, the plate 226 can include four chambers 230. In other embodiments, the plate 226 can include five chambers 230. In a other embodiments, the plate 226 can include six chambers or more 230.

FIG. 3C is an enlarged top view of a chamber 230 of the carrier plate 226 with a cell transplant 206 illustrated by a dotted line which is positioned within the chamber 230. As illustrated in this figure, the body 228 of the carrier plate 226 includes an overhanging structure made up of a pair of tabs 232. The overhanging structure defined by the tabs 232 defines a slot (not visible in FIG. 3C) underneath the tabs 232. The slot can be sized and shaped to retain a portion of the cellular transplant from migrating from the chamber 230 when, for example, the carrier plate 226 is positioned within the carrier housing (not shown) and the entire cellular transplant carrier 208 is positioned in a tube with solution (e.g., preservation medium) therein. As can be seen by the dotted line of the cell transplant 206, a portion of the circular portion of the cellular transplant would fit within the slot and underneath the overhanging structure of the tabs 232.

As illustrated in FIG. 3C, the chambers are defined on a side edge 234 of the carrier plate 226. The chamber 230 further includes a recessed portion 236 defined on the side edge 234 of the carrier plate 226. The tab portion of the cell transplant 206 would overhang the recessed portion 236 when the cell transplant 206 is received within the chamber 230. The recessed portion 236 permits a gripping portion of a surgical instrument to grasp the cellular transplant 206 by the tab portion and remove it from the carrier plate 226 with minimal disruption of the cells.

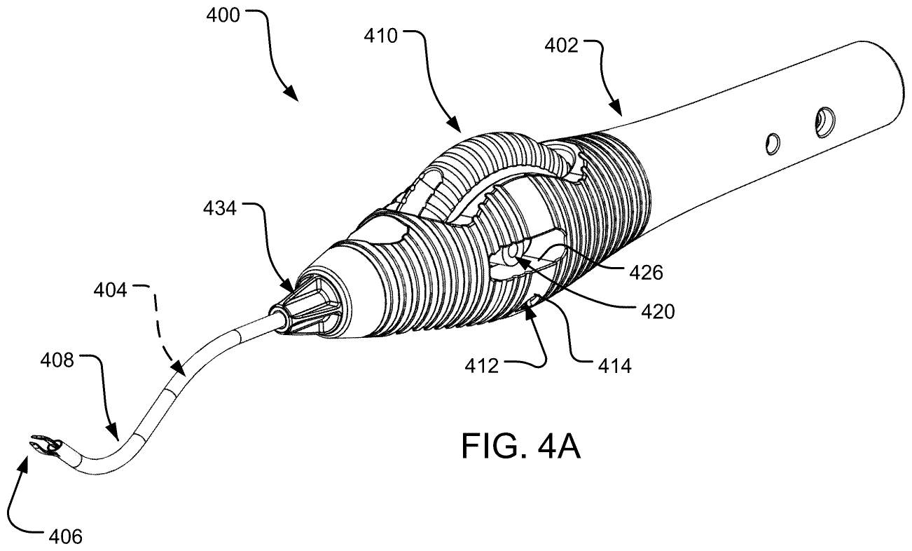

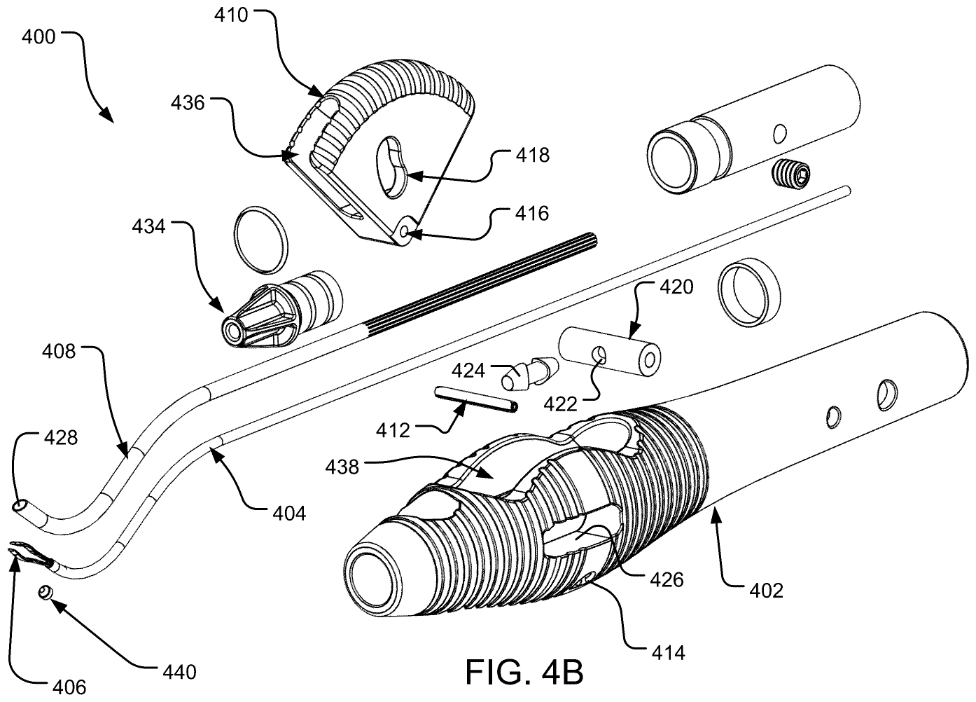

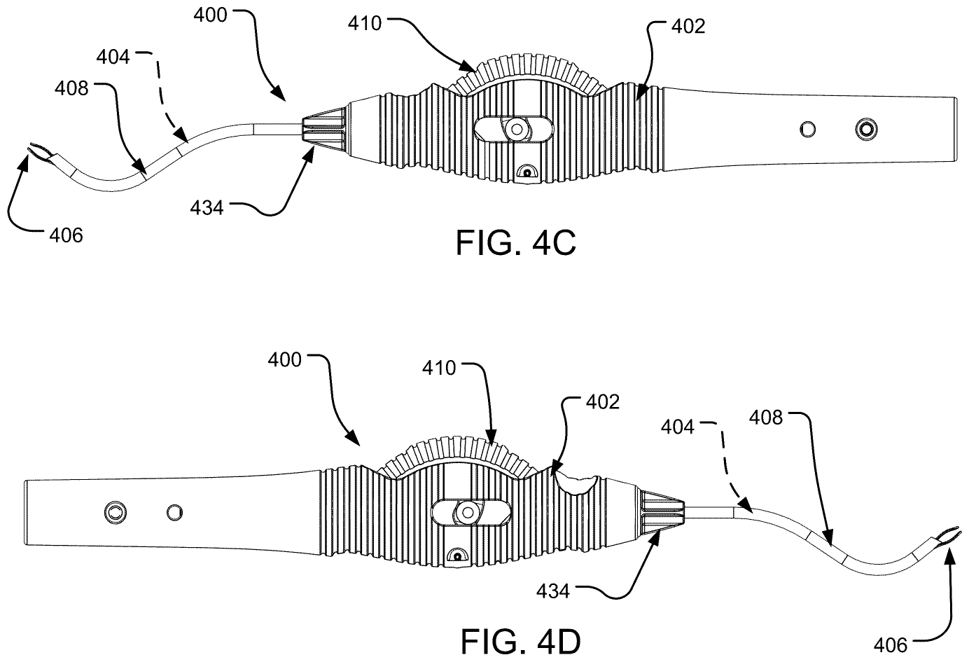

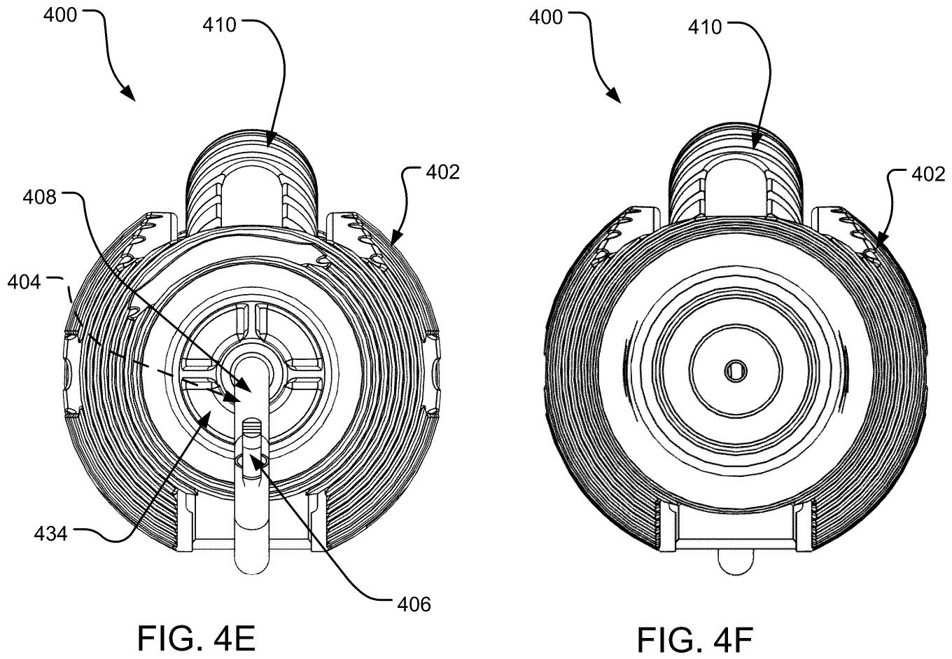

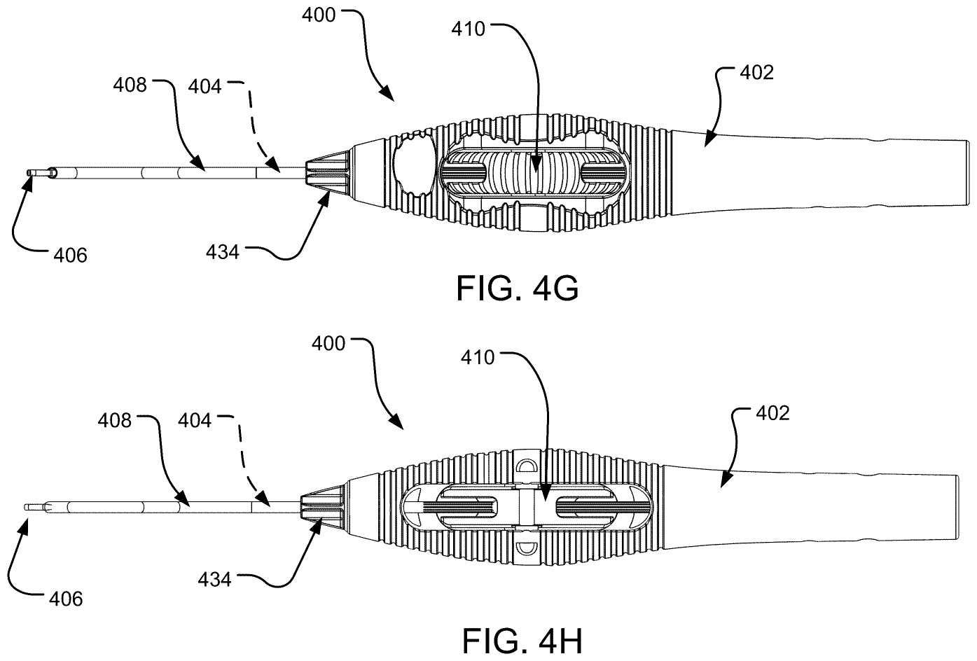

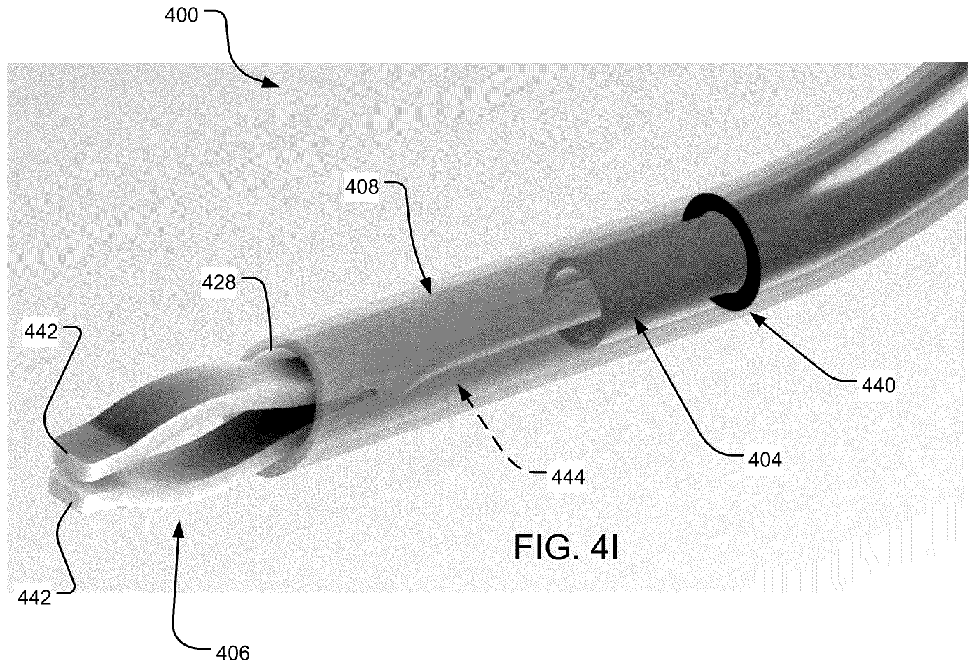

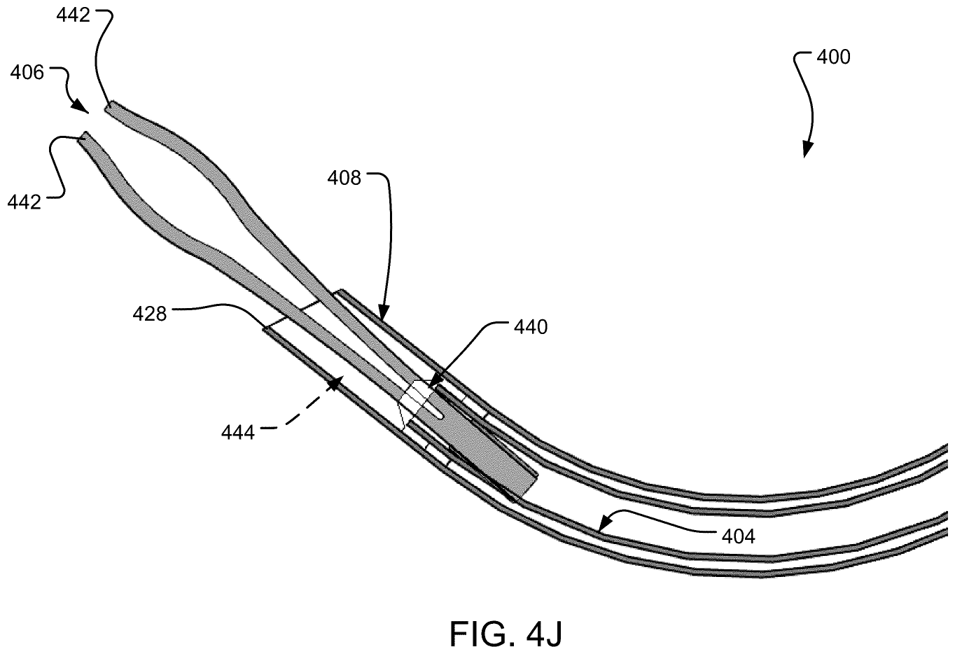

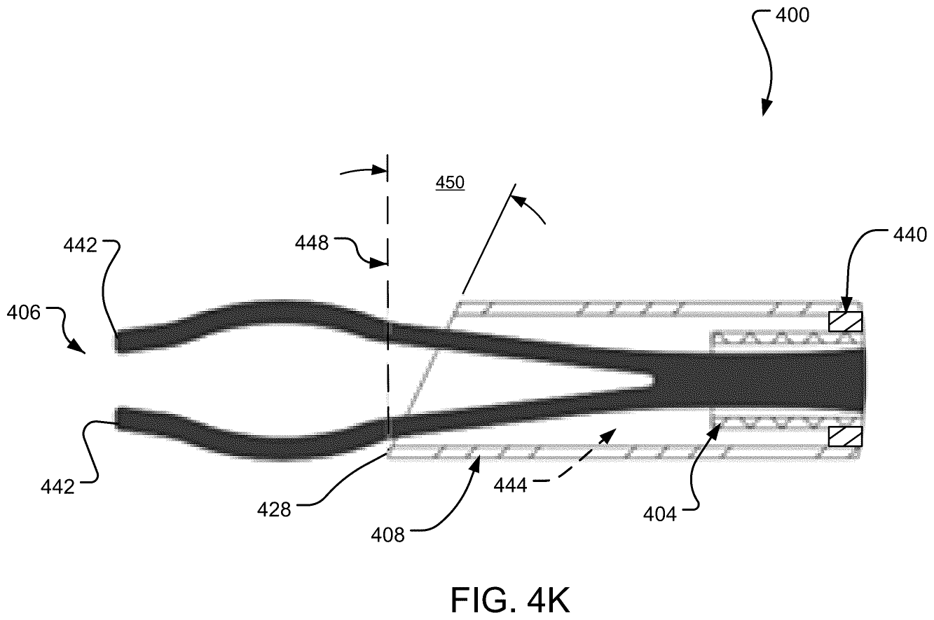

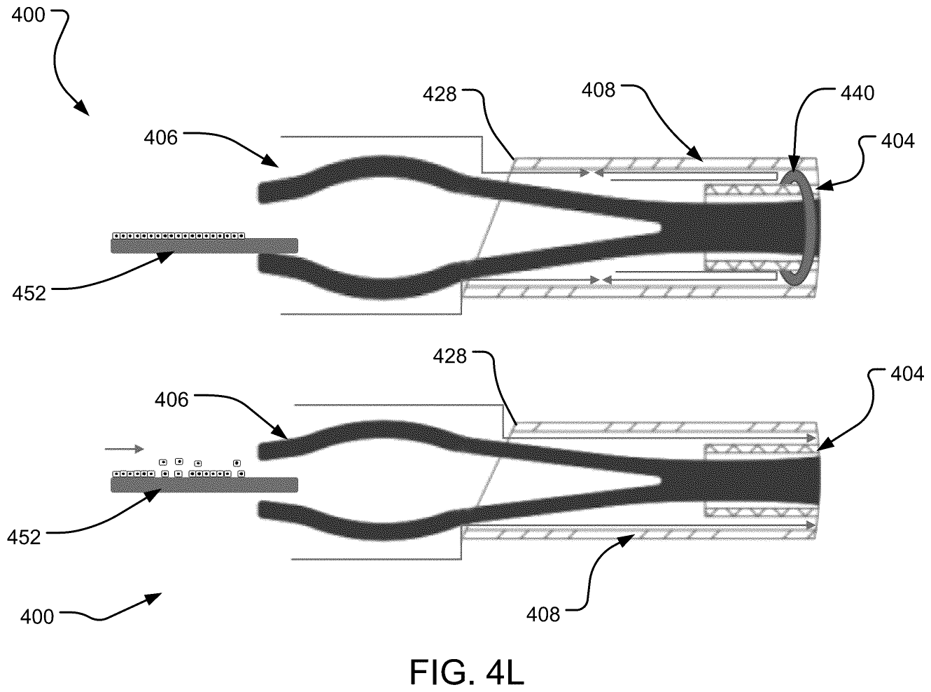

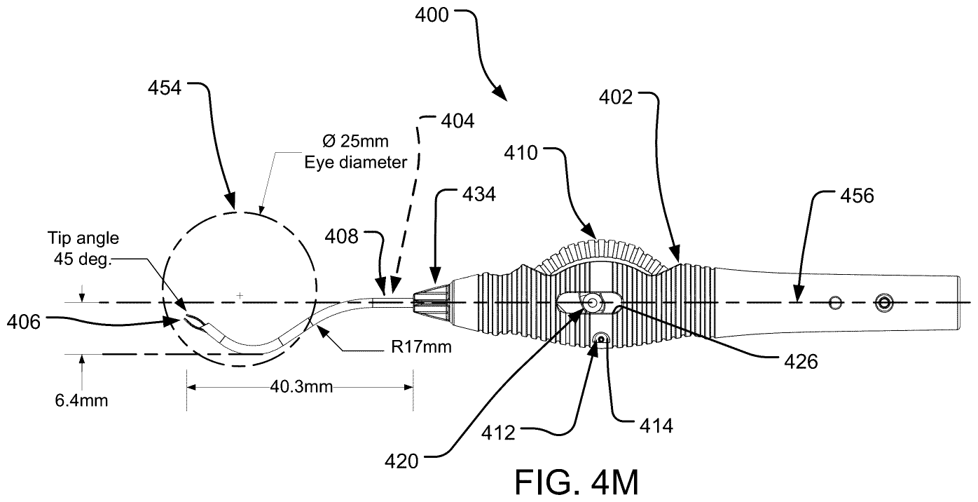

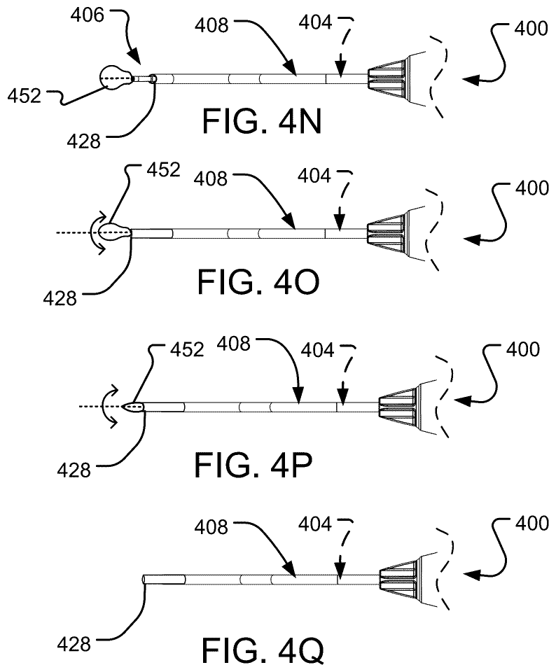

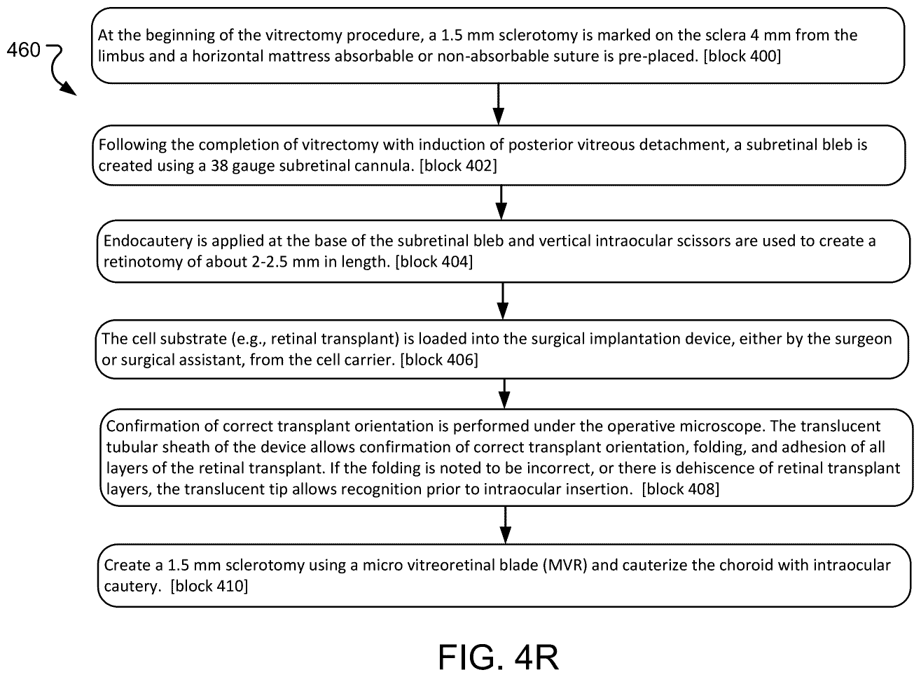

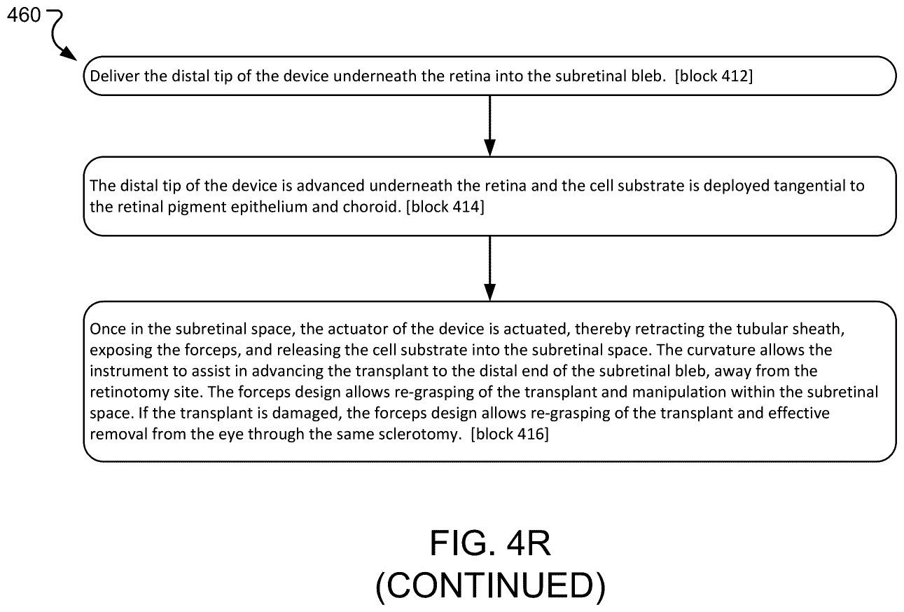

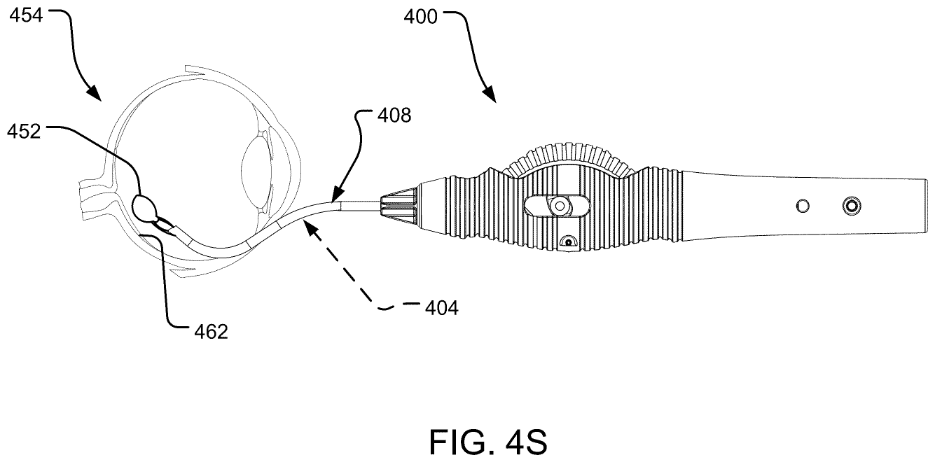

One embodiment includes devices for implantation of cellular transplants and methods of using the same is described in reference to FIGS. 4A-4R. Reference is made to FIGS. 4A-4H, which are, respectively, a front isometric view, a front isometric exploded view, a first side view, a second side view, a front/distal view, a back/proximal view, a top view, and a bottom view of a surgical implantation device 400.

As illustrated in FIGS. 4A-4H, the surgical implantation device 400 includes a handpiece 402, a shaft 404, a forceps or micro-jaws 406 at the distal end of the shaft 404, a tubular sheath 408 that slides over the shaft 404 and forceps 406, and an actuator or wheel 410 that is moveably coupled to the handpiece 402 via a pin 412. In certain examples, the pin 412 can include a spring that biases the pin 412 in a certain direction. For example, the pin 412 can bias the actuator 410 in a distal position with the tubular sheath 408 positioned over the forceps 406. In certain examples, the pin 412 can bias the actuator 410 in the opposite, proximal direction. The actuator 410 is pivotable about the pin 412 which is also received within a transverse opening 414 in the handpiece 402.

As illustrated in the enlarged view of FIG. 4B, the actuator 410 includes a corresponding transverse opening 416 in which the pin 412 is received therethrough. The actuator 410 also includes an arcuate opening 418 that extends transversely therethrough. A pin clamp 420 is positioned through the arcuate opening 418. The pin includes a through hole 422 for receiving the tubular sheath 408 and the shaft 404 therethrough. The pin clamp 420 is secured to the tubular shaft 408 via an adhesive 424, for example. The tubular sheath 404 is, thus, moveably coupled to the shaft 404 permitting the tubular sheath 408 to slide over and relative to the shaft 404 via movement (pivoting) of the actuator 410.

The handpiece 402 also includes a transverse opening 426 in the form of an elongated slot for receiving the pin claim 420 therein. The transverse opening 426 permits linear translation of the pin clamp 420 in a distal-proximal direction. As can be understood from the figures, when the actuator 410 is pivoted or rotated proximally (towards the back) about the pin 412, the pin clamp 420 translates linearly within the transverse opening 426 in a proximal direction. The pin clamp 420, which is coupled to the tubular sheath 408, translates the tubular sheath 408 proximally relative to the shaft 404 and handpiece 402. As will be described subsequently, proximal retraction of the tubular sheath 408 causes the distal tips of the forceps 406 to extend out from the distal opening 428 of the tubular sheath 408. In this way, the forceps 406 remain positioned at the same place relative to the handpiece 402 during operation with only the tubular sheath 408 being proximally and distally moved. The forceps 406 are non-locking structures having a pair of distal tips and a hinge opposite the tips. The forceps 406 are biased such that when they are positioned within the lumen of the tubular sheath 408, the inner walls of the sheath 408 force the forceps 406 together or closed. In opposite configuration, when the forceps 406 are extended out the distal opening 428 of the sheath 408, the forceps 406 spring outward and open.

Referring to FIG. 4B, the shaft 404 is fixed in position at its proximal end via a set screw 430 that extends through the handpiece 402 and a proximal end hub 432, which is insertable into the proximal end of the handpiece 402. The set screw 430 contacts the shaft 404 when tightened in order to affix the position of the shaft 404 relative to the handpiece 402.

Opposite the proximal end hub 432 is a distal insertion adaptor 434 at the distal end of the handpiece 404. The distal insertion adaptor 434 maintains the alignment of the shaft 404 along a longitudinal axis of the handpiece 402 and includes a distal opening for the shaft 404 and tubular sheath 408 to extend therethrough.

The actuator 410 includes distal and proximal slots 436 to permit the actuator 410 to pivot while allowing the shaft 404 and tubular sheath 408 to extend into the slots 436. The handpiece 402 also includes an elongated opening 438 for receiving the actuator 410 therein. A seal 440 is also illustrated in FIG. 4B. The seal 440 can be a ring and be sized to fit over the shaft 404 and up against the inner wall of the tubular sheath 408. The seal 440 is designed to restrict fluid from entering the device 400 via the tubular sheath 408 when the tubular sheath 408 is inserted into a subject's eye during surgery. During surgery, the eye can be pressurized with fluid. Without a seal 440, the pressurized fluid would fill the space in the tubular sheath 408. Additionally, the turbulent flow around the distal tips of the forceps 406 caused by fluid flowing into the tubular sheath 408 could cause disruption to the cells on the cellular transplant which is held by the forceps 406.

Referring to FIGS. 4I and 4J, which are, respectively, an enlarged isometric view of the surgical implantation device 400 and an enlarged cross-sectional side view of the surgical implantation device 400, the tubular sheath 408 is translucent in order to improve visualization during surgery. Both figures depict the device 400 in a deployed state. That is, the distal tips 442 of the forceps 406 are positioned outside the distal opening 428 of the tubular sheath 408. In this state, the forceps 406 are opened and designed to release its grasp on the cellular transplant 206 (not shown). As seen in the figures, the distal tips 442 are not touching in the deployed state. Also as seen in the figure, the forceps 406 are coupled to the distal end of the shaft 404 and the seal 440 is positioned between the shaft 404 and the inner wall of the tubular sheath 408.

A non-deployed state would be when the distal tips 442 of the forceps 406 are positioned within the lumen (inside of the tubular sheath 408) 444 of the tubular sheath 408. Distal advancement of the tubular sheath 408 relative to the shaft 404 and the forceps 406 would cause the device 400 to transition from a deployed state to a non-deployed state. Conversely, proximal retraction of the tubular sheath 408 relative to the shaft 404 and the forceps 406 could cause the device 400 to transition from a non-deployed state to a deployed state. In the non-deployed state, the distal tips 442 of the forceps 406 could be forced together via compression by the inner walls of the tubular sheath 408. In this state, the forceps 406 could grasp the cellular transplant 206 (not shown) and house the same within the lumen 444 of the tubular sheath 408. In this way, the only way to employ the grasping and releasing of the forceps 406 is via movement of the tubular sheath 408 relative to the shaft 404.

FIGS. 4I, 4J, and 4K illustrate the beveled tip of the distal opening 428 of the tubular sheath 408. FIG. 4K depicts an up-close cross-sectional side view of the forceps 406 and tubular sheath 408 of the surgical implantation device 400. As illustrated in FIG. 4K, the distal opening 428 of the tubular sheath 408 is beveled at an angle 450 relative to a perpendicular axis 448 of the tubular sheath 408. In certain examples, the beveled angle 450 is about 15 degrees. In certain examples, the beveled angle 450 is about 25 degrees. In certain examples, the beveled angle 450 is about 35 degrees. In certain examples, the beveled angle 450 is about 45 degrees.

FIG. 4L depicts side views of a device 440 with a seal 440 at the top and a device 400 without a seal. As illustrated in these figures, both devices 400 are in the deployed state with the distal tips of the forceps 406 exposed out from the distal opening 428 of the tubular sheath 408 so as to deploy the cellular transplant 452 for implantation at an implantation site. These views show the fluid flowpath with arrows with the seal 440 in place (top) and without it in place (bottom). These flowpaths would be exhibited during a surgical procedure when the tubular sheath 408 is positioned within a patient's eye with the eye being pressurized with fluid. In the top image, the seal 440 blocks the fluid flow from entering the lumen of the tubular sheath 408. In this manner, there is less turbulent flow to the fluid, which in turn causes minimal disruption of the cells on the cell substrate 452. In the bottom image, the fluid flowpath goes into the lumen of the tubular sheath 408 until the pressure equalizes. Thus, the fluid can rush past the cellular transplant 452 and cause the cells to be removed from the cellular transplant 452 and/or die in the process.

FIG. 4M illustrates a side view of the surgical implantation device 400 illustrating various exemplary dimensions of the device 400. The device 400 is positioned relative to a diagram of an eye 454 to demonstrate the relative size of the device 400. Of note, the shaft 404 includes a linear section immediately distal to the handpiece 402. The shaft 404 then transitions to a curved section or portion that follows a compound curve. The compound curve means that there are two radii of curvature associated with the bend of the shaft 404. That is, the proximal portion of the bend of the shaft 404 has a first radius of curvature, and the distal portion of the bend of the shaft 404 has a second radius of curvature. The first and second radius of curvature are different than each other. The curve of the shaft 404 is designed to generally follow the arc of the posterior pole of the eye. As seen in the figure, the forceps 406 are generally positioned along the longitudinal axis 456 of the handpiece 402 and the linear portion of the shaft 404. This provides improved visualization of the cell transplant attached to the forceps 406.

Reference is now made to FIGS. 4N-4Q, which illustrate the grasping of a cellular transplant 452 by the forceps 406 of the surgical implantation device 400 and the housing of the cellular transplant 452 in the lumen of the tubular sheath 408. FIG. 4N depicts a top view of the device 400 grasping a cellular transplant 452 having a shape as illustrated in FIG. 3A. As illustrated in this figure, the forceps 406 are extended out from the distal opening 428 of the tubular sheath 408. The forceps 406 are closed just enough to grasp the cellular transplant 452 by the tubular sheath 408 distally advancing relative to the shaft 404 and forceps 406, which remain stationary. As illustrated in FIG. 4O, the tubular sheath 408 is beginning to distally advance relative to the shaft 404 and the forceps 406. Using these methods, the cellular transplant 452 comes into contact with the beveled distal opening 428 of the tubular sheath 408. The cellular transplant 452 begins to concavely fold along the dotted longitudinal axis. That is, the cellular transplant 452 begins to conform to a tube, matching a shape of the inner walls of the tubular sheath 408. FIG. 4P illustrates further distal advancement of the tubular sheath 408 and further folding of the cellular transplant 452 into a tube-shape. FIG. 4Q illustrates the cell transplant being fully housed within the tubular sheath 408. This figure illustrates the non-deployed state of the device 400 and cellular transplant.

With the cellular transplant loaded into the device 400, the device can be utilized in a surgical procedure. To transition the device 400 from the non-deployed state to the deployed state, the steps illustrated in FIGS. 4N-4Q can be reversed. For example, the tubular sheath 408 can be proximally retracted relative to the forceps 406 and shaft 404, which can expose the cellular transplant 452 out of the distal opening 428 of the tubular sheath 408. This can cause the cellular transplant 452 to unfold from a cylindrical shape to a generally flat shape or orientation.

FIG. 4R is a flowchart illustrating an exemplary method 460 of performing a surgery using the surgical implantation device 400 described herein. The method 460 is merely exemplary and may not include all steps to a particular procedure. As illustrated in the flowchart of FIG. 4R, at [block 400], to begin the vitrectomy procedure, a 1.5 millimeter (mm) sclerotomy is marked on the sclera 4 mm from the limbus and a horizontal mattress absorbable or non-absorbable suture is pre-placed. At [block 402] and following the completion of vitrectomy with induction of posterior vitreous detachment, a subretinal bleb is created using a 38 gauge subretinal cannula. At [block 404], endocautery is applied at the base of the subretinal bleb and vertical intraocular scissors are used to create a retinotomy of about 2-2.5 mm in length. At [block 406], the cellular transplant (e.g., retinal transplant) is loaded into the surgical implantation device, cither by the surgeon or surgical assistant, from the cell carrier. At [block 408], under the operative microscope, the correct orientation of the cellular transplant within tubular sheath of the surgical implantation device is confirmed. The translucent tubular sheath of the device allows confirmation of correct transplant orientation, folding, and adhesion of all layers of the retinal transplant. If the folding is noted to be incorrect, or there is dehiscence of retinal transplant layers, the translucent tip allows recognition prior to intraocular insertion. At [block 410], create a 1.5 mm sclerotomy using a micro vitreoretinal blade (MVR) and cauterize the choroid with intraocular cautery. The beveled tip of the tubular sheath allows for ergonomic introduction of the instrument through the sclerotomy. At [block 412], deliver the distal tip of the device underneath the retina into the subretinal bleb. The intraluminal gasket/seal located behind the forceps prevents egress of intraocular fluid from the pressurized vitreous chamber through the lumen of the tubular sheath of the device. This minimizes turbulent fluid dynamics within the instrument lumen, to minimize any trauma or dislocation of the retinal transplant. At [block 414], the distal tip of the device is advanced underneath the retina and the cell implant is deployed tangential to the retinal pigment epithelium and choroid. The double curvature of the shaft of the device allows proper ergonomic positioning of the device outside of the eye. The curvature near the tip allows the instrument to be directed parallel to the posterior pole of the eye (FIG. 4S). The curved instrument tip prevents vector forces from being directed downward towards the retinal pigment epithelium (RPE), thereby minimizing trauma to the RPE and choroid. That is, the relatively less abrasive curved portion of the shaft of the instrument is closest to the RPE and would, therefore, minimize trauma to the RPE and choroid upon contact. At [block 416], once in the subretinal space, the actuator of the device is actuated, thereby retracting the tubular sheath, exposing the forceps, and releasing the cell transplant into the subretinal space. The curvature allows the instrument to assist in advancing the transplant to the distal end of the subretinal bleb, away from the retinotomy site. The forceps design allows re-grasping of the transplant and manipulation within the subretinal space. If the transplant is damaged, the forceps design allows re-grasping of the transplant and effective removal from the eye through the same sclerotomy.

FIG. 4S illustrates a side view of the surgical implantation device 400 positioned relative to an eye 454. As illustrated in this figure, the curvature of the shaft 404 permits the device 400 to be positioned generally parallel to the posterior pole 462 of the eye 454. As stated previously, the shaft 404 has a double curvature where it angles downward and then angles upwards at the distal end. The shaft 404 generally matches the contour of the curvature of the eye 454. With the device 400 positioned generally parallel to the posterior pole 462 of the eye 454, visualization of the device is improved during the procedure, and it provides for an ergonomic trajectory for delivery of the cell transplant 452.

### EXAMPLES

It is understood that the examples and embodiments described herein are for illustrative purposes only and are not intended to limit the scope of the claimed invention. It is also understood that various modifications or changes in light the examples and embodiments described herein will be suggested to persons skilled in the art and are to be included within the spirit and purview of this application and scope of the appended claims.

Exemplary devices disclosed herein provide solutions to preventing, reducing the risk of and treating currently incurable retinal degenerative conditions where transplantation of retinal cell transplant is one therapy. These devices and methods apply to pre-clinical and clinical phases of cell-based regenerative therapies. In certain disclosure related to devices herein, a transplantation device is disclosed that is the first to provide an all-in-one foldable technology that for example, minimizes incision size, controlled delivery speed, no fluid reflux, curved translucent tip, usability of loading and in vivo reloading, and ergonomic handle. This transplantation device can be combined with a customizable transplant carrier device disclosed herein to ensure viability of cell transplants during storage and transport, proper orientation of cell transplants, and usability of loading into the transplantation device.

In another example, a customizable transplant carrier device is designed to ensure viability of cell transplants during storage and transport, proper orientation of cell substrate, and usability of loading into a transplantation device. This transplantation delivery device is the first to provide all-in-one foldable technology that minimizes incision size, controlled delivery speed, no fluid reflux, curved translucent tip, usability of loading and in vivo reloading, and ergonomic handle. Combined these two technologies offer an all-encompassing surgical kit for implantation of cell-based transplants.

What is claimed is:

1. A device for implantation of a cellular transplant into a target region of a subject, comprising:
a handpiece;
a shaft anchored with the handpiece and extending distally therefrom, the shaft comprising a proximal end, a distal end opposite the proximal end, a curved portion, and forceps at the distal end thereof, the forceps comprising a pair of distal tips configured to releasably grasp the cellular transplant; and
a tubular sheath movably coupled to and extending distally from the handpiece, the tubular sheath comprising a distal opening, and a lumen configured to slidably receive the shaft and the forceps therein,
wherein, in a non-deployed state, the distal tips of the forceps are positioned within the lumen of the tubular sheath, and, in a deployed state, the distal tips of the forceps are positioned at least partially outside the distal opening of the tubular sheath.

2. The device of claim 1, wherein retraction of the tubular sheath relative to the handpiece transitions the device from the non-deployed state to the deployed state.

3. The device of claim 2, wherein, in the non-deployed state, the forceps are configured to grasp the cellular transplant, and, in the deployed state, the forceps are configured to release the cellular transplant.

4. The device of claim 2, wherein, in the non-deployed state, the forceps are compressed by the tubular sheath and grasp the cellular transplant.

5. The device of claim 2, wherein the cellular transplant is configured to transition from a folded orientation to a generally flat orientation when the device transitions from the non-deployed state to the deployed state.

6. The device of claim 1, wherein the distal opening of the tubular sheath is beveled.

7. The device of claim 6, wherein the tubular sheath is beveled by an angle of about 15 to about 45 degrees, or about 25 to about 45 degrees, or about 35 to about 45 degrees, or about 45 degrees.

8-10. (canceled)

11. The device of claim 1, further comprising a seal positioned between the shaft and an inner wall of the tubular sheath, the seal preventing backflow of fluid into the lumen of the tubular sheath.

12. The device of claim 1, wherein the forceps are non-locking forceps further comprising a hinge opposite the distal tips.

13. The device of claim 1, wherein the curved portion of the shaft comprises at least one of the following: a compound curve; is configured to angle the forceps at an upward angle relative to the handpiece; matches a profile of a posterior pole of an eye; and orients the forceps at about a 45° angle relative to a longitudinal axis of the handpiece.

14-16. (canceled)

17. The device of claim 1, further comprising an actuator coupled with the tubular sheath and moveably coupled to the handpiece, whereby actuation of the actuator is configured to cause retraction of the tubular sheath relative to the handpiece to transition the device from the non-deployed state to the deployed state.

18. The device of claim 17, wherein the actuator comprises a rotatable member in which rotation thereof causes retraction of the tubular sheath relative to the handpiece.

19. The device of claim 1, wherein the shaft is rigid, and the tubular sheath is flexible to conform to a shape of the shaft as it slides over the shaft.

20. The device of claim 1, wherein the tubular sheath is at least one of translucent and wherein the tubular sheath comprises a Teflon material.

21. (canceled)

22. The device of claim 1, wherein the cellular transplant comprises at least one of a retinal pigment epithelium (RPE) cellular transplant, a cellular transplant including retinal cells, and a retina-RPE complex transplant.

23. A surgical kit comprising:
a device according to claim 1; and
instructions for use.

24. The surgical kit of claim 23, further comprising a trephine configured to cut the cellular transplant, wherein the trephine is configured to cut the cellular transplant in a shape comprising a circular portion and a tab portion extending outward from a perimeter of the circular portion.

25. (canceled)

26. The surgical kit of claim 24, further comprising a carrier device comprising a carrier housing and a carrier plate configured to be inserted into the carrier housing, the carrier device configured to house the cellular transplant.

27. (canceled)

28. A method of implanting a cellular transplant into an eye of a subject, the method comprising:
providing a device according to claim 1;
using the forceps of the device to grasp the cellular transplant;
inserting a portion of the tubular sheath of the device into the eye of the subject; and
releasing the cellular transplant inside the eye of the subject.

29. (canceled)

30. The method of claim 28, wherein releasing the cellular transplant inside the eye comprises retracting the tubular sheath relative to the handpiece so as to unfold the cellular transplant and release the forceps from grasping the cellular transplant inside the eye of the subject; optionally, wherein the cellular transplant is released within a subretinal region of the eye of the subject.

31. (canceled)

32. The method of claim 28, wherein the cellular transplant comprises at least one of a retinal pigment epithelium (RPE) cellular transplant, a retina-RPE complex transplant, a neural retina cellular transplant, and a neural retina-RPE cellular transplant.

33. (canceled)

34. The method of claim 28, wherein the subject has a retinal condition comprising one or more of a retinal degenerative condition or a retinal injury, a retinal condition causing loss or damage to the retinal pigment epithelium (RPE) or outer neurosensory retina, advanced dry age-related macular degeneration (AMD), retinitis pigmentosa, Stargardt disease, Best disease, Sorsby's fundus dystrophy, Doyne honeycomb retinal dystrophy, retinal trauma, or a combination thereof.

35. (canceled)
